(19) United States
(12) Patent Application Publication
(10) Pub. No.: US 2024/0327786 A1
(43) Pub. Date: Oct. 3, 2024

(54) NON-MAMMALIAN AQUATIC ANIMAL CELLS AND EXTRACTS

(71) Applicant: Shah Caviar Limited, Burscough (GB)

(72) Inventor: Kenneth Benning, Burscough (GB)

(21) Appl. No.: 18/579,454

(22) PCT Filed: Jul. 14, 2022
(86) PCT No.: PCT/GB2022/051823

§ 371 Date: Jan. 15, 2024

(30) Foreign Application Priority Data
Jul. 14, 2021 (GB) 2110124.1
Feb. 2, 2022 (GB) 2201345.2

(51) Int. Cl. C12N 5/075; A23L 17/30

(52) U.S. Cl. CPC C12N 5/0609; A23L 17/30; C12N 2501/11; C12N 2501/115; C12N 2501/13; C12N 2501/235; C12N 2501/31

(57) ABSTRACT

The invention relates to an in vitro method of cultivating cells from a non-mammalian aquatic animal, to cells derived from a non-mammalian aquatic animal, and to products containing the same.

The present invention relates to an in vitro method of cultivating cells from non-mammalian aquatic animals, in particular germ cells from non-mammalian aquatic animals, in particular oocytes from non-mammalian aquatic animals. The method employs in vitro culturing of primordial germ cells (PGCs) from non-mammalian aquatic animals to induce differentiation into germ cells and harvesting the germ cells, in particular oocytes. The present invention also relates to PGCs from non-mammalian aquatic animals which have been immortalised to enhance or prolong their ability to differentiate into germ cells, in particular oocytes. These immortalised PGCs from non-mammalian aquatic animals may be used in the in vitro methods of the invention for producing germ cells, in particular oocytes. The present invention also relates to immortalised oocytes from non-mammalian aquatic animals and to immortalised spermatozoa from non-mammalian aquatic animals. Immortalised stage 1 oocytes from non-mammalian aquatic animals may also be used in the in vitro methods of the invention for producing oocytes. The present invention also relates to germ cells from non-mammalian aquatic animals, in particular oocytes, which are produced by methods of the invention. Oocytes of the invention (including oocytes produced by methods of the invention) are typically “Stage 1” oocytes. The present invention also relates to products comprising oocytes of the invention, typically Stage 1 oocytes of the invention, such as cosmetic, nutritional, and nutraceutical products.

Herein, the term “caviar” corresponds to traditional use of the term in relation to salt-treated, unfertilized eggs (oocytes) or roe from ovaries of female sturgeons, and which are full-size “Stage 3”-“Stage 5” sturgeon oocytes. Herein, the term “caviar” also embraces full-size oocytes from ovaries of other types of non-mammalian aquatic animal. Like sturgeon caviar, caviar from ovaries of other types of non-mammalian aquatic animal are typically ‘full size’ oocytes, typically “Stage 3”-“Stage 5” oocytes.

Non-mammalian aquatic animals include sturgeons and other types of fish, such as salmon, lumpfish, hake, trout, capelin, steelhead, whitefish, carp, pollock (e.g. Alaska pollock), herring (e.g. Atlantic herring), tobiko, imitation tobiko, cod (e.g. Atlantic cod), tuna, whitefish, catfish, shad, orange roughy, mullet, mako shark, perch (e.g. pike perch), jawless fish, armoured fish, spiny shark, cartilaginous fish, bony fish, ray-finned fish or lobe-finned fish. Non-mammalian aquatic animals also include echinoderms, such as sea urchin or sea cucumber. Non-mammalian aquatic animals also include crustacea, such as crab or lobster.

In a preferred embodiment, the non-mammalian aquatic animal is a fish.

Herein, the term “caviar” typically refers to oocytes from ovaries of non-mammalian aquatic animals whose oocytes are consumed by humans. Herein, the term “caviar” also embraces oocytes from ovaries of non-mammalian aquatic animals whose oocytes are consumed by non-human mammals, such as domestic pets e.g. cats and dogs.

Caviar is generally regarded as a luxury food owing to its difficulty of production and perishable nature, and is prized for qualities including flavour, shape, colour and texture.

The most highly prized caviar is from sturgeon. Sturgeon is the common name for a large number of fish species of the family Acipenseridae, including the genera Acipenser, Huso, Scaphirhynchus and Pseudoscaphirhynchus (for more information see: www.sturgeonweb.co.uk). In the context of sturgeon caviar, the term “caviar” is used more exclusively to refer to two species commonly connected with caviar production, Acipenserand Huso. For example Beluga caviar derived from wild Beluga sturgeon (Huso huso) has traditionally been much prized, but other sturgeon species are also well-known for sturgeon caviar production and are increasingly farmed. These include for example Siberian sturgeon (Acipenser baerii), now being farmed for the first time for sturgeon caviar production in the UK by Exmoor Caviar, and other Acipenser species.

As well as being a food, often a luxury food, caviar is widely recognised for its nutritional and cosmetic benefits. Desirable properties associated with caviar include enhanced cellular metabolism, stimulation of collagen production by fibroblasts, protection against oxidative stress (and associated improved maintenance of cell membranes) and hydration of the skin. Caviar is also recognised for its desirable profile of proteins, amino and fatty acids, lipids, vitamins and minerals. The desirable properties of caviar have resulted in widespread use of caviar and caviar extracts in applications which extend far beyond its traditional use as a food, including as a luxury food. Nowhere is this more pronounced than in cosmetics and nutritional applications, where caviar and caviar extracts (particularly sturgeon caviar and sturgeon caviar extracts) are now being used e.g. in skin creams, and to supplement pet foods and vodka.

Despite considerable and growing demand for caviar and caviar extracts, caviar production remains labour intensive and slow, particularly for sturgeon caviar.

By way of example, a key limiting factor in the production of sturgeon caviar is the many years that female sturgeons take to reach reproductive maturity. Female Siberian sturgeons commonly take 8-10 years to become mature and produce eggs; females of some species, e.g. Beluga sturgeon, take far longer, e.g. around 20 years or more. To reduce this time, some hybrid crosses have been achieved, e.g. male Huso huso sturgeon have been crossed with faster maturing species such as Acipenser sterlet or baerii to provide hybrid species for farming, but even hybrid crosses take several years to reach reproductive maturity.

Whilst farming of sturgeons addresses the ecological problem of relying on wild sturgeon for sturgeon caviar production and enables sturgeon caviar production more widely, there remains for sturgeon caviar producers the problem of the need for careful timing of harvesting of the roe, preferably very shortly before spawning, coupled with need for roe sacks to be quickly processed once extracted to avoid deterioration of the roe. This is still a delicate task usually done by hand. The roe sacks are normally gently rubbed across a sieve, either a nylon mesh or stainless steel sieve, whereby the roe eggs are separated from the membrane of the roe sack and pass through the sieve to be collected. Running cold water may be employed to aid the roe release and separation. Salt will be added to the separated roe according to requirement for taste and to aid preservation. A stabilizer may be additionally added such as E285 stabilizer. The roe, which is now sturgeon caviar, is packed into containers with airtight sealing, normally under vacuum, for refrigerated storage. The sturgeon caviar may be subsequently re-packaged in smaller quantities.

High global demand is not limited to sturgeon caviar. Caviar from other types of non-mammalian aquatic animals is highly desired for a wide variety of applications, ranging from cosmetics to nutrition. Whilst these other types of caviar are typically less expensive than sturgeon caviar, the environmental impact imposed by their harvesting is also devastating on rivers and oceans—by way of example, in a single year (2018), Russian fisheries harvested 22,200 metric tonnes of salmon roe alone. Such high demand for caviar is even more acutely felt by fish populations which are already highly depleted, such as cod and lumpfish. Moreover, the ethical concerns surrounding sacrifice of non-mammalian aquatic animals apply just as they do for sturgeon, and likewise limit the potential market for other types of caviar. Ultimately, despite advancements made in the caviar industry, production remains slow, and caviar remains comparatively expensive. Established caviar production methods are also hampered by the requirement to sacrifice non-mammalian aquatic animals during caviar harvesting, which limits the potential commercial market for products which contain caviar and caviar extracts.

There exists a need in the art for a product which possesses highly desirable properties associated with caviar, but is faster, cheaper and more ethical to produce, and can be produced in sufficient quantities to meet the rapidly growing demand. There exists a particular need for a product which possesses highly desirable properties associated with caviar, and which helps alleviate the considerable pressure placed on fish populations, particularly populations which are already highly depleted, such as cod and lumpfish. There also exists a need in the art for a method of producing such a product.

The present invention is based on the surprising realisation that many requirements of caviar as a food, often as a luxury food (e.g. flavour, shape, colour and texture) are largely irrelevant to its use in other applications (e.g. in nutritional or cosmetic products). Herein, “nutritional products” include products consumed for their nutritional value, but do not include traditional caviar (full size oocytes from ovaries of non-mammalian aquatic animals). Indeed, the appearance and texture of caviar (so desired by caviar afficionados) is often destroyed during preparation (e.g. of nutritional or cosmetic products) and can even be undesirable (e.g. in face creams). Despite this, caviar and caviar extracts remain a gold standard for many applications. Consequently, the high cost of many existing products which contain caviar and caviar extracts (but are not caviar itself) is largely driven by requirements imposed by the food industry, particularly the luxury food industry, even though many such requirements are irrelevant to other applications. Moreover, current production of products which contain caviar and caviar extracts typically requires sacrifice of non-mammalian aquatic animals, such as fish, which raises ethical questions and limits their potential commercial market.

The present invention is also based on the surprising discovery that “Stage 1” oocytes from non-mammalian aquatic animals (which are at an earlier developmental stage than caviar) possess highly desirable properties that are intrinsic to caviar (such as enhanced cellular metabolism, stimulation of collagen production by fibroblasts, protection against oxidative stress (and associated improved maintenance of cell membranes) and hydration of the skin; as well as a desirable profile of proteins, amino and fatty acids, lipids, vitamins and minerals). Like caviar, Stage 1 oocytes of the invention are also highly suitable for use in e.g. nutritional or cosmetic products.

Although Stage 1 oocytes from non-mammalian aquatic animals possess highly desirable properties that are intrinsic to caviar from non-mammalian aquatic animals, to the best of the inventors' knowledge, Stage 1 oocytes from non-mammalian aquatic animals have not previously been consumed directly as a foodstuff (unlike caviar), and neither have they been used in nutritional or cosmetic products. Indeed, when seeking to provide highly desirable properties that are intrinsic to caviar from non-mammalian aquatic animals, the market remains focussed on caviar itself.

Stage 1 oocytes of the invention are visibly different from caviar, most notably in terms of their size. For example, most stage 1 fish oocytes are typically less than 200 μM in diameter, whereas e.g. sturgeon caviar oocytes are typically 1000-1200 μM in diameter, sockeye salmon caviar oocytes are typically around 5600 μM in diameter, and chum salmon caviar oocytes are typically around 8300 μM in diameter. Consistent with this size difference (which is not constrained to fish oocytes), Stage 1 oocytes of the invention have a different appearance and texture to caviar, and so lack e.g. the visual and mouthfeel qualities of caviar as food, particularly caviar as a luxury food. The inventors believe that the market focus on caviar (such as Stage 3-Stage 5 fish oocytes; or extracts thereof) has been so strong that alternatives which fail to meet the requirements placed on caviar as a food, particularly as a luxury food, have not even been contemplated.

The present invention advantageously ‘uncouples’: (A) the highly desirable properties that are intrinsic to caviar (e.g. for use in nutritional and cosmetic products), from (B) requirements placed on caviar as a food, particularly as a luxury food. Unlike caviar, Stage 1 oocytes from non-mammalian aquatic animals may be produced in vitro. As compared to caviar:
i) Stage 1 oocytes of the invention may be produced more quickly than caviar (no waiting for non-mammalian aquatic animals e.g. fish, to reach reproductive maturity, including no waiting for oocytes to reach (at least) Stage 3);
ii) Stage 1 oocytes of the invention may be produced more cheaply (costs associated with in vitro production are far lower than fishing or farming, particularly sturgeon fishing or farming); and
iii) Stage 1 oocytes of the invention avoid ethical concerns and provide a greater commercial market (no involvement of non-mammalian aquatic animals e.g. fish, in their production).

To produce germ cells from non-mammalian aquatic animals (e.g. germ cells from fish, such as sturgeon) in commercially viable quantities, the inventors overcame a number of technical challenges. By way of background, fish cells have been manipulated in laboratories since the 1970s, beginning with fertilised oocytes of Zebrafish (Danio rerio) as a model organism for fundamental biological research (Streisinger et al. 1981). More recently, fish cells have been cultivated and manipulated in laboratories to study fish reproduction with a view to developing technologies to preserve endangered species (Wong et al. 2013). In most fish species, including sturgeon, the head kidney is an organ analogous to the mammalian adrenal gland, which secretes important hormones controlling growth and development (Geven and Klaren, 2017). Ciba et al. (2008) isolated cells from a head kidney tissue harvested from a 1-year-old Siberian sturgeon (Acipenser baerii) and transferred them to tissue culture vessels in growth media. Head kidney-derived cells were successfully passaged in standard growth media for 1 year. The recovered ovarian cells were confirmed as being germ cells by the presence of the germ line-specific protein, vasa. These ovarian cells were transplanted to the abdominal cavity of the larvae of ‘sterlet’ sturgeon (Acipenser ruthenus) where they were observed to proliferate, from less than 10 cells to over 100 cells, over a 90-day period.

Dabry's sturgeon (Acipenser dabryanus), alternatively known as the Yangtze sturgeon, Chiangjiang sturgeon or river sturgeon, is a sturgeon native to China. Xie et al. (2019) recovered cells from the gonads of a 2-year-old Dabry's sturgeon. Of the gonad-derived cells, non-germ cells tended to adhere to the surface of plastic culture flasks, allowing recovery of germ cells which remained in suspension. Germs cells were cultivated for up to 40 days in serum-free media, with a tenfold increase in cell number observed over that time.

Currently there is no method for long term cultivation of cells from non-mammalian aquatic animals e.g. fish, that permit rapid growth of cells to high numbers in a time scale of 1-7 days at low cost, along with continued cycles of cryopreservation and cryorevival of cells which preserve their growth properties over extended time periods.

Prior to the present invention, there was a need in the art for methods whereby cells harvested from a non-mammalian aquatic animal, such as a fish, are rendered immortal, cultivated in a manner that their growth in number over time is rapid, can be triggered to differentiate into a desired cell type and can retain these properties throughout unlimited cycles of cryopreservation and cryorevival. This need is met by the present invention which provides in vitro methods for producing germ cells from a non-mammalian aquatic animal, in particular oocytes.

Germ cells (e.g. immortalised germ cells) and PGCs (e.g. immortalised PGCs) of the invention are ideally-suited to production in culture medium (e.g. as adherent cell cultures or suspension cell cultures), which enables their efficient cell production at low cost. Unexpectedly, the invention provides isolated germ cells (e.g. immortalised germ cells) and PGCs (e.g. immortalised PGCs) which grow in suspension cell culture (herein “suspension” cells), and methods for producing the same. Suspension cells are particularly desirable for bulk production and batch harvesting, and are especially advantageous for industrial and research applications. As compared to adherent cells, suspension cells are easier to passage, they do not require enzymatic or mechanical dissociation, and may be easily scaled-up.

According to the invention, enzyme-based cell separation methods may be used to harvest cells from tissues from non-mammalian aquatic animals e.g. fish. Procedures are then applied to expand harvested, ovary-derived cells in liquid culture which utilise methods for sorting and isolation of desired cell types such as adherent/suspension subculturing, bead-based methods or fluorescence-activated cell sorting (FACS). Suspension cells are particularly desirable. Serial cell passaging may then be performed until spontaneous immortalisation is observed. Limiting dilution techniques may be employed to obtain clonally derived populations of immortalised cells e.g. immortalised fish cells. Where oocytes are desired, clonally derived populations (‘clones’) are screened to identify those clones which possess markers and phenotypes of oocytes and exhibit rapid cell division. Where spermatozoa are desired (e.g. for use in artificial fertilisation), clones are screened to identify those clones which possess markers and phenotypes of spermatozoa and exhibit rapid cell division. Where PGCs are desired (e.g. fish PGCs, such as sturgeon PGCs), clones are screened to identify those clones which possess markers and phenotypes of PGCs and exhibit rapid cell division.

Master and working cell banks of immortalised cell clones from non-mammalian aquatic animals e.g. fish, are established and stored under liquid nitrogen and in −80° C. freezer units. Clones with desirable characteristics are typically conditioned to serum-free and, finally, chemically defined media whilst being monitored for retention of required characteristics such as growth performance. An ovary analogue environment within which to direct oocyte differentiation and enlargement may be established in the growth media and its components, including recombinant proteins.

Growth media supplements which favour oocyte in vitro differentiation include e.g. epidermal growth factor (e.g. about 25 ng/mL), basic fibroblast growth factor (e.g. about 25 ng/mL), human chorionic gonadotropin (e.g. about 5 IU/mL), pregnant mare serum gonadotropin (e.g. about 2 IU/mL), glial cell line-derived neurotrophic factor (e.g. about 25 ng/mL) and leukemia inhibitory factor (e.g. about 25 ng/mL).

The invention provides an isolated stage 1 germ cell from a non-mammalian aquatic animal. In some embodiments, the isolated stage 1 germ cell is an oocyte.

The invention also provides an immortalised germ cell from a non-mammalian aquatic animal. In some embodiments, the immortalised germ cell is an oocyte. In some embodiments, the germ cell is a stage 1 oocyte. In some embodiments, the germ cell is a stage 2 oocyte. In some embodiments, the germ cell is a stage 3 oocyte, a stage 4 oocyte or a stage 5 oocyte.

In some embodiments, the immortalised germ cell is a spermatozoa.

The invention also provides an immortalised primordial germ cell (PGC) from a non-mammalian aquatic animal.

In some embodiments, the germ cell is a suspension germ cell. In some embodiments, the immortalised germ cell is an immortalised suspension germ cell. In some embodiments, the PGC is a suspension PGC. In some embodiments, the immortalised PGC is an immortalised suspension PGC.

In some embodiments, the germ cell is an adherent germ cell. In some embodiments, the immortalised germ cell is an immortalised adherent germ cell. In some embodiments, the PGC is an adherent PGC. In some embodiments, the immortalised PGC is an immortalised adherent PGC.

The invention also provides use of a germ cell of the invention (e.g. an immortalised germ cell of the invention) as an intermediate in the production of a higher stage oocyte. Thus, in one embodiment, the invention provides use of a stage 1 oocyte as an intermediate in the production of a stage 2 oocyte, a stage 3 oocyte, a stage 4 oocyte, or a stage 5 oocyte.

The invention also provides use of a PGC of the invention (e.g. an immortalised PGC of the invention) as an intermediate in the production of a germ cell, optionally an oocyte e.g. a stage 1 oocyte, a stage 2 oocyte, a stage 3 oocyte, a stage 4 oocyte, or a stage 5 oocyte.

The invention also provides an extract of a germ cell of the invention or an extract of an immortalised PGC of the invention.

The invention also provides a cosmetic product comprising a germ cell of the invention, an immortalised PGC of the invention, or an extract of the invention. The invention also provides a cosmetic product comprising an isolated oocyte from a non-mammalian aquatic animal such as a stage 1 oocyte, a stage 2 oocyte, a stage 3 oocyte, or a stage 4 oocyte, or an extract thereof.

The invention also provides a nutritional product comprising a germ cell of the invention, an immortalised PGC of the invention, or an extract of the invention. The invention also provides a nutritional product comprising an isolated oocyte from a non-mammalian aquatic animal such as a stage 1 oocyte, a stage 2 oocyte, a stage 3 oocyte, or a stage 4 oocyte, or an extract thereof.

The invention also provides a nutraceutical product comprising a germ cell of the invention, an immortalised PGC of the invention, or an extract of the invention. The invention also provides a nutraceutical product comprising an isolated oocyte from a non-mammalian aquatic animal such as a stage 1 oocyte, a stage 2 oocyte, a stage 3 oocyte, or a stage 4 oocyte, or an extract thereof.

In some embodiments, the extract, the cosmetic product or the nutritional product, further comprises salt. In some embodiments, the salt is a sodium salt e.g. sodium chloride.

The invention also provides an in vitro method of producing suspension germ cells from a non-mammalian aquatic animal, the method comprising: (a) culturing a mixture of cells comprising germ cells from a non-mammalian aquatic animal in growth media until the mixture of cells reaches confluency; (b) separating suspension cells from the cultured mixture of cells to provide a mixture of suspension cells; and (c) optionally separating suspension germ cells from the mixture of suspension cells.

In some embodiments, separating suspension germ cells from the mixture of suspension cells comprises screening for genetic markers for germ cells.

In some embodiments, the mixture of cells is a purified mixture of germ cells from a non-mammalian aquatic animal.

In some embodiments, the method further comprises culturing the germ cells in the presence of an epigenetic modifier, optionally wherein the epigenetic modifier is selected from valproic acid and butyric acid.

In some embodiments, the method further comprises culturing the germ cells in the presence of feeder cells such as zebra fish liver cells and/or rainbow trout fibroblasts.

In some embodiments, the method further comprises culturing the germ cells in the presence of additional media supplements such as recombinant carp growth hormone, vitellogenin, salmon gonadotropin releasing hormone (sGnRHa)—(Ovaprim), luteinizing hormone or gonadotropin releasing hormone.

In some embodiments, the method further comprises culturing the germ cells in the presence of soluble Sturgeon roe sac material and/or Sturgeon serum (blood).

In some embodiments, the germ cell is selected from a stage 1 germ cell, optionally a stage 1 oocyte, and a spermatozoa.

The invention also provides an in vitro method of producing suspension PGCs from a non-mammalian aquatic animal, the method comprising: (a) culturing a mixture of cells comprising PGCs from a non-mammalian aquatic animal in growth media until the mixture of cells reaches confluency; (b) separating suspension cells from the cultured mixture of cells to provide a mixture of suspension cells; and (c) optionally separating suspension PGCs from the mixture of suspension cells.

In some embodiments, separating suspension PGCs from the mixture of suspension cells comprises screening for genetic markers for PGCs.

In some embodiments, the mixture of cells is a purified mixture of PGCs from a non-mammalian aquatic animal.

In some embodiments, the method further comprises culturing the PGCs in the presence of an epigenetic modifier, optionally wherein the epigenetic modifier is selected from valproic acid and butyric acid.

In some embodiments, the method further comprises culturing the PGCs in the presence of feeder cells such as zebra fish liver cells and/or rainbow trout fibroblasts.

In some embodiments, the method further comprises culturing the PGCs in the presence of additional media supplements such as recombinant carp growth hormone, vitellogenin, salmon gonadotropin releasing hormone (sGnRHa)—(Ovaprim), luteinizing hormone or gonadotropin releasing hormone.

In some embodiments, the method further comprises culturing the PGCs in the presence of soluble Sturgeon roe sac material and/or Sturgeon serum (blood).

The invention also provides an in vitro method of producing isolated stage 1 oocytes from a non-mammalian aquatic animal, the method comprising: (a) culturing a PGC from a non-mammalian aquatic animal to induce differentiation into germ cells; and optionally (b) separating stage 1 oocytes from the PGCs. In some embodiments, the invention provides an in vitro method of producing isolated stage 1 oocytes from a non-mammalian aquatic animal, the method comprising culturing a PGC from a non-mammalian aquatic animal to induce differentiation into germ cells. In some embodiments, the invention provides an in vitro method of producing isolated stage 1 oocytes from a non-mammalian aquatic animal, the method comprising: (a) culturing a PGC from a non-mammalian aquatic animal to induce differentiation into germ cells; and (b) separating stage 1 oocytes from the PGCs. In some embodiments, methods of producing isolated stage 1 oocytes further comprise a step of washing isolated stage 1 oocytes e.g. to remove culture media. In some embodiments, the PGCs are suspension PGCs. In some embodiments, the isolated stage 1 oocytes are isolated suspension stage 1 oocytes.

The invention also provides an in vitro method of producing isolated spermatozoa from a non-mammalian aquatic animal, the method comprising: (a) culturing a PGC from a non-mammalian aquatic animal to induce differentiation into germ cells; and optionally (b) separating spermatozoa from the PGCs. In some embodiments, the invention provides an in vitro method of producing isolated spermatozoa from a non-mammalian aquatic animal, the method comprising culturing a PGC from a non-mammalian aquatic animal to induce differentiation into germ cells. In some embodiments, the invention provides an in vitro method of producing isolated spermatozoa from a non-mammalian aquatic animal, the method comprising: (a) culturing a PGC from a non-mammalian aquatic animal to induce differentiation into germ cells; and (b) separating spermatozoa from the PGCs. In some embodiments, methods of producing isolated spermatozoa further comprise a step of washing isolated spermatozoa e.g. to remove culture media. In some embodiments, the PGCs are suspension PGCs. In some embodiments, the isolated spermatozoa are isolated suspension spermatozoa.

The invention also provides an in vitro method of producing an extract of an isolated stage 1 oocyte from a non-mammalian aquatic animal, the method comprising: (a) culturing a PGC from a non-mammalian aquatic animal to induce differentiation into germ cells; (b) optionally separating stage 1 oocytes from the PGCs; and (c) processing the oocytes to provide an extract. In some embodiments, the invention provides an in vitro method of producing an extract of an isolated stage 1 oocyte from a non-mammalian aquatic animal, the method comprising: (a) culturing a PGC from a non-mammalian aquatic animal to induce differentiation into germ cells; and (b) processing the oocytes to provide an extract. In some embodiments, the invention provides an in vitro method of producing an extract of an isolated stage 1 oocyte from a non-mammalian aquatic animal, the method comprising: (a) culturing a PGC from a non-mammalian aquatic animal to induce differentiation into germ cells; (b) separating stage 1 oocytes from the PGCs; and (c) processing the oocytes to provide an extract. In some embodiments, methods of producing an extract of an isolated stage 1 oocyte further comprise a step of washing isolated stage 1 oocytes e.g. to remove culture media. In some embodiments, the PGCs are suspension PGCs. In some embodiments, the isolated stage 1 oocyte is an isolated suspension stage 1 oocyte.

In some embodiments, the in vitro method comprises separating germ cells from PGC(s) from a non-mammalian aquatic animal prior to separating oocytes from the PGCs. In some embodiments, PGC is an immortalised PGC.

In some embodiments, the invention provides an in vitro method of producing isolated stage 1 oocytes from a non-mammalian aquatic animal, the method comprising culturing immortalised stage 1 oocytes to induce proliferation of stage 1 oocytes. In some embodiments, the method further comprises a step of washing stage 1 oocytes e.g. to remove culture media.

In some embodiments, methods for producing isolated stage 1 oocytes (e.g. immortalised stage 1 oocytes) further comprise a step of producing isolated suspension stage 1 oocytes (e.g. immortalised suspension stage 1 oocytes) as described herein.

In some embodiments, methods for producing PGCs (e.g. immortalised PGCs) further comprise a step of producing suspension PGCs (e.g. immortalised suspension PGCs) as described herein.

In some embodiments, methods for producing isolated stage 1 oocytes (e.g. immortalised stage 1 oocytes) further comprise a step of producing isolated adherent stage 1 oocytes (e.g. immortalised adherent stage 1 oocytes) as described herein.

In some embodiments, methods for producing PGCs (e.g. immortalised PGCs) further comprise a step of producing adherent PGCs (e.g. immortalised adherent PGCs) as described herein.

The invention also provides a method of producing a cosmetic product, the method comprising combining: (a) a germ cell of the invention, an immortalised PGC of the invention, or an extract of the invention; with (b) ingredients for a cosmetic product.

The invention also provides a method of producing a nutritional product, the method comprising combining: (a) a germ cell of the invention, an immortalised PGC of the invention, or an extract of the invention; with (b) ingredients for a nutritional product.

The invention also provides a method of producing a nutraceutical product, the method comprising combining: (a) a germ cell of the invention, an immortalised PGC of the invention, or an extract of the invention; with (b) ingredients for a nutraceutical product.

### Nutritional Products

A nutritional product of the invention comprises a germ cell of the invention, an immortalised PGC of the invention, and/or an extract of the invention. Nutritional products of the invention are typically consumed as a food.

In some embodiments, the nutritional product comprises one or more intact germ cells of the invention. In some embodiments, the nutritional product comprises one or more intact immortalised PGCs of the invention.

In some embodiments, the nutritional product comprises one or more disrupted germ cells of the invention. In some embodiments, the nutritional product comprises one or more disrupted immortalised PGCs of the invention. Disruption may be by any means, such as mechanical (e.g. by shear force), heating (e.g. during cooking) and/or lysis.

In some embodiments, the nutritional product is fresh. In one embodiment, the nutritional product is frozen. In some embodiments, the nutritional product is formulated in brine. In some embodiments, the nutritional product is smoked. In some embodiments, the nutritional product is canned. In some embodiments, the nutritional product is provided in a tube. In some embodiments, the nutritional product is spreadable. In some embodiments, the nutritional product is vacuum packed.

In some embodiments, the nutritional product is sealed.

In some embodiments, the nutritional product comprises a colourant.

In some embodiments, the nutritional product is sealed and heat-processed e.g. pasteurised.

In some embodiments, the nutritional product comprises smoked germ cells of the invention. In some embodiments, the nutritional product comprises smoked immortalised PGC of the invention. In some embodiments, the nutritional product comprises smoked extract of the invention.

In some embodiments, the nutritional product comprises sugar-salted germ cells of the invention. In some embodiments, the nutritional product comprises sugar-salted immortalised PGC of the invention. In some embodiments, the nutritional product comprises sugar-salted extract of the invention.

In some embodiments, the nutritional product comprises dried and optionally salted germ cells of the invention. In some embodiments, the nutritional product comprises dried and optionally salted immortalised PGC of the invention. In some embodiments, the nutritional product comprises dried and optionally salted extract of the invention.

In some embodiments, the nutritional product comprises oil, typically vegetable oil. The nutritional product may further comprise one or more of bread, lemon juice, garlic and pepper. In some embodiments, the nutritional product is taramasalata.

In some embodiments, the nutritional product comprises sugar-salted germ cells, sugar-salted immortalised PGCs and/or sugar-salted extract of the invention, and oil, typically vegetable oil. The nutritional product may further comprise one or more of dill and potato flour.

In some embodiments, the nutritional product comprises compressed germ cells, compressed immortalised PGCs and/or compressed extract of the invention.

In some embodiments, the nutritional product comprises dried and salted germ cells, dried and salted immortalised PGCs and/or dried and salted extract of the invention. In some embodiments, the nutritional product comprises compressed dried and salted germ cells, compressed dried and salted immortalised PGCs and/or compressed dried and salted extract of the invention. In some embodiments, a thin layer of beeswax is applied to the compressed dried and salted germ cells, to the compressed dried and salted immortalised PGCs and/or to the compressed dried and salted extract of the invention. In some embodiments, the nutritional product is bottarga or poutargue.

In some embodiments, the nutritional product comprises a germ cell of the invention, an immortalised PGC of the invention, and/or an extract of the invention, encapsulated within a bead or a pearl. In one embodiment, the bead or pearl is a gel. In one embodiment, the bead or pearl comprises sodium alginate.

Methods of producing beads or pearls comprising sodium alginate are well known in the art and are frequently referred to in molecular gastronomy as ‘spherification’ methods. Typically, an “alginated liquid” comprising sodium alginate is mixed with (typically dripped into) a solution comprising a calcium salt, typically calcium chloride or calcium glucate lactate. Each drop of the alginated liquid typically forms a ‘sphere’ in the solution comprising a calcium salt. During a reaction time of a few seconds to a few minutes, calcium in the solution comprising a calcium salt causes formation of a thin flexible skin or shell around the alginated liquid drops. In one embodiment of the invention, a germ cell of the invention, an immortalised PGC of the invention, and/or an extract of the invention is mixed with sodium alginate to provide an alginated liquid, and then mixed with (typically dripped into) a solution comprising a calcium salt, typically calcium chloride or calcium lactate. In one embodiment, the alginated liquid further comprises a salt (typically 1-6% salt, such as 2-5%, 2-4% or 3% salt). In one embodiment, the alginated liquid further comprises a salt (typically 1-6% salt, such as 2-5%, 2-4% or 3% salt) and E285 (borax). When present, the borax is typically at a concentration of 0.1-1%, such as 0.2-0.8%, 0.3-0.7% or 4%). In some embodiments, the alginated liquid comprises a colourant.

In one embodiment, a germ cell of the invention, an immortalised PGC of the invention, and/or an extract of the invention is mixed with sodium alginate (0.5%) for 3 mins to provide desirable consistency, and then added dropwise to a cold water bath containing calcium lactate (0.5%). The incubation period is adjusted depending on the desired hardness of the beads or pearls. In some embodiments, salt and optionally borax is then added to the pearls.

### Summary of Key Terms

“Stage 1 oocytes” are also known as “pre-vitellogenesis stage”, “early previtellogenic” or “primary oocyte stage” oocytes. Stage 1 oocytes are gonad cells e.g. from fish, which have lost stem cell markers. Stage 1 oocytes start off very small (typically 7-10 μM) and do not yet contain yolk. The number increases through mitotic division and at the end of this stage the oocytes increase their size to approximately 200 μM.

“Stage 2 oocytes” are also known as “endogenous vitellogenesis stage” or “mid-previtellogenic”. Within this stage the yolk of the oocyte is formed. The origin of the yolk in this stage is the oocyte itself.

“Stage 3 oocytes” are also known as “exogenous vitellogenesis” or “late-previtellogenic”. During this stage, the oocyte increases to its final size of 1000-1200 μM (1-1.2 mm). During this phase yolk formation in the oocyte increases. A large nucleus (0.2 mm) is clearly visible. The oocytes in this stage are also called “ripe eggs”. They remain in this stage until environmental factors trigger ovulation.

“Stage 4 oocytes” are also known as “ovulated eggs” or “vitellogenic”. Ovulated eggs are ovulated or liberated from the ovarian tissue. During ovulation, the yolk accumulates into one droplet, the eggs become transparent and the nucleus migrates completely to the outer-side (animal pole) of the egg.

“Stage 5 oocytes” are also known as “atretic eggs” or “postvitellogenic”. These are not well ovulated and will be re-absorbed or recycled by the non-mammalian aquatic animal, e.g. fish. Stage 5 oocytes are distinguished by their “flushy” appearance.

Herein, an “isolated” cell is a cell which has been isolated from its natural environment. Herein, an “isolated” cell also refers to a cell which has been produced by a non-natural process, such as an immortalised cell (or proliferation product of an immortalised cell).

Herein, “separating” oocytes or spermatozoa from PGCs refers to substantially extracting oocytes or spermatozoa from media which contains PGCs.

A “germ cell” refers to an oocyte or a spermatozoa.

A “primordial germ cell” (PGC) is the primary undifferentiated stem cell type that will differentiate into spermatozoa or oocytes.

Whilst it will be appreciated that the establishment of cell line(s) might initially require one or more cells obtained directly from a non-mammalian aquatic animal, as used herein, a cell “from a non-mammalian aquatic animal” is typically a cell which is derived from (e.g. proliferated from) cell(s) obtained directly from a non-mammalian aquatic animal.

An “immortalised cell” is a cell which proliferates indefinitely and can thus be cultured for long periods of time. Methods of immortalising cells (mammalian and non-mammalian) are well known in the art and include genetically modified cells (“GM” cells; such as cells comprising an exogenous hTERT gene) and cells which have been immortalised by non-GM techniques (e.g. as described in the Examples). Non-GM techniques are preferred for use according to the present invention, such as passaging cells until ‘crisis’ (which is when the majority of cells die or stop growing); monitoring for growing cells which have become spontaneously immortal; and isolating those cells. A non-GM method of immortalisation is detailed in the Examples section “Immortalisation of cells derived from fish tissues”.

Cell “extracts” contain cell extractives and are typically formulated with an excipient such as water and/or glycerin. In some embodiments, a cell extract contains homogenised cells. In some embodiments, a cell “extract” contains cell fractions, such as a soluble fraction or an insoluble fraction. In some embodiments, the soluble fraction is hydrophobic. In some embodiments, the soluble fraction is lipophilic. In some embodiments, the cell extract is a solution comprising at least 0.01%, at least 0.05%, at least 1%, at least 2%, at least 5%, at least 10%, at least 15%, at least 20%, at least 25%, at least 30%, at least 40%, at least 50%, at least 60%, at least 70%, at least 80%, at least 90% or at least 95% cell extractive. In some embodiments, the cell extract is formed entirely of homogenised cells.

A “cosmetic product” refers to a product intended to be rubbed, poured, sprinkled, or sprayed on, introduced into, or otherwise applied to the human body for cleansing, beautifying, promoting attractiveness, or altering the appearance. Included in this definition are products such as skin moisturizers, perfumes, lipsticks, fingernail polishes, eye and facial makeup preparations, shampoos, permanent waves, hair colours, toothpastes, and deodorants, as well as any material intended for use as a component of a cosmetic product. In one embodiment, the cosmetic product is a finished cosmetic product.

A “nutritional product” refers to a product which either supplements the nutrition or provides part or all of the daily nutritional requirements. It includes intravenous or oral nutrition that can provide the nutrition. Nutritional products may be for human consumption. Nutritional products may be for animal consumption (e.g. pet food).

A “nutraceutical product” refers to a product derived from a food source that provides extra health benefits in addition to the basic nutritional value found in food.

“Suspension cells” are single cells or small aggregates of cells which multiply while suspended in liquid culture medium. Suspension cells are typically grown in agitated liquid culture medium.

“Adherent cells” are single cells or small aggregates of cells which multiply while attached to a surface. Adherent cells are typically grown in static liquid medium. Adherent cells typically require a surface to form a monolayer.

### BRIEF DESCRIPTION OF THE DRAWINGS

FIG. 1: Morphological assessment of primary sturgeon ovarian cells passage 0.

Microscopic analysis of primary sturgeon ovarian cells after 10 days in culture. The cell culture comprises a mixture of adherent cells (highlighted by a white asterisk) and suspension cells. Scale bar is 100 μm.

FIG. 2: Morphological assessment of primary sturgeon ovarian cells passage 1.

Microscopic analysis of primary sturgeon ovarian cells after one passage. The majority of cells are small, round suspension cells. Scale bar is 100 μm.

FIG. 3: Morphological assessment of primary sturgeon ovarian cells passage 3.

Microscopic analysis of primary sturgeon ovarian cells after three passages. The cell culture contains a homogenous population of small round suspension cells with a diameter of ˜6-7 μm. Scale bar is 100 μm.

FIG. 4: Morphological assessment of primary sturgeon ovarian cells passage 3 in round-bottom plate.

Microscopic analysis of primary sturgeon ovarian cells after three passages. When cultured in a 96 well round bottom plate, the suspension cells gather in the middle of the plate and display a high degree of homogeneity. Scale bar is 100 μm.

### EXAMPLES

The invention will now be described with reference to the following non-limiting examples.

### Isolation of Cells From Fish Tissues

Sturgeon fish were held in a recirculating aquaculture system at a mean temperature of 10° C. Gonads from female fish, aged 4-40 years, average ovary/body weight 4.2/910 g, are used for all procedures. Ovaries exhibiting maturity stage II were extracted and transferred in samples of 1.5 mL volume to 15 mL Falcon tubes containing 5 mL of phosphate-buffered saline (PBS) solution (Sigma-Aldrich P4417), Hank's balanced salt solution (Sigma-Aldrich H6648), or Leibovitz medium (L-15; Sigma-Aldrich L5520) with differing concentrations of trypsin (T; Sigma-Aldrich T1426) and collagenase (C; Sigma-Aldrich C0130): (1) 0.1% T and 0.1% C, (2) 0.3% T, (3) 0.1% T, (4) 0.3% C, or (5) 0.1% C. The tissues were incubated for 2 hours at 23° C. Osmolality of media was adjusted to that of blood plasma of the fish used (mean, 238 mOsm kg⁻¹), and pH was adjusted to 8. The obtained suspension was filtered with 50-mm filter (Partec, Germany), to collect larger oocytes and debris, and 1% bovine serum albumen (BSA, Sigma-Aldrich A7511) and 40 mg mL⁻¹ DNAse (AppliChem A3778) added. The 15 mL Falcon tube with ovary cell suspension was centrifuged at 500×g for 30 minutes at 4° C. The cell pellet was then resuspended in 0.3 mL of growth media without enzymes. The yield and viability of cells obtained from each combination were evaluated by hemocytometer (Burker's cell counting chamber) and Live/Dead Cell Double Staining Kit (Sigma-Aldrich, 04511), respectively. The number of cells was counted in 20 squares of the hemocytometer in three repetitions under a microscope (Olympus BH2) at ×100 magnification. For the viability determination, at least 500 cells per sample were recorded using the Olympus IX83 microscope.

The cell suspension was then loaded onto a discontinuous Percoll (Sigma-Aldrich P1644) concentration gradient 5%, 10%, 20%, 30%, 40%, and 50% in PBS and centrifuged at 800×g for 30 minutes. Each cell fraction was removed from the gradient and transferred to a tube, diluted in PBS 1:10, and centrifuged again at 800×g for 30 minutes. The pellets were resuspended in PBS and examined using immuno-fluorescence labeling.

### Characterisation and Ranking of Cells Isolated From Fish Tissues

Proteins were extracted from cells with a lysis buffer with typical constituents of 8M urea, 2M thiourea, 4% CHAPS, 10% wt/vol isopropanol, 0.1% wt/vol Triton X-100, and 100-mM dithiothreithol (DTT) containing protease inhibitors, 100-mM PMSF, 1 mg mL¹ pepstatin A and 5 mg mL⁻¹ leupeptin. The Bradford protein assay was applied to determine protein concentration of the samples. For SDS-PAGE, samples, 25 mg of proteins per lane, were resuspended in a buffer containing 65-mM Tris, 10% (v:v) glycerol, 2% (wt/vol) SDS, and 5% (v:v) beta-mercaptoethanol and boiled for 3 minutes at 95° C. Proteins were separated on 12% SDS gel. After electrophoresis, the gels were placed on polyvinyl difluoride membranes (Bio-Rad, USA) and electrically transferred. The membranes were blocked by incubation with 5% (wt/vol) skim milk in TBST (0.1% Tween-20, 20-mM Tris, 500-mM NaCl at pH 7.6) for 1 hour at 20° C. The membranes were incubated for 12 hours at 4° C. in 5% BSA-TBST containing DDX4, a rabbit polyclonal antibody (GTX116575, GeneTex), as the primary antibody specific for germline cells and subsequently incubated with Horseradish Peroxidase-conjugated goat antirabbit immunoglobulin G (1:3000 in 3% BSA-TBST) for 1 hour at 20° C. The reacted proteins were revealed with 3,30,5,50-tetramethylbenzidine liquid substrate.

DDX4 is a marker for germline cells. The percentage of DDX4 antibody positive cells collected from 10%, 20%, 30%, 40%, and 50% Percoll solution was 83.6%, 74.6%, 54.2%, 21.9%, and 10.4% for ovarian cells, respectively, whereas dissociated control somatic cells showed no fluorescent signal after immunolabeling with DDX4. Cell solutions from the 10% to 30% Percoll fractions were used for further passaging to obtain polyclonal fish populations. Approximately 1 million DDX4-positive cells were isolated from a given fish in this manner. Ranking is performed according to strength of DDX4 expression. Higher levels of DDX4 expression are favoured (higher ranking) because it corresponds c to pronounced germ cell-like characteristics. Lower ranking cells (with lower DDX4 expression levels) are also maintained and characterised for prior differentiation into oocytes.

### Production of Suspension Cells

A culture of primary sturgeon ovarian cells was centrifuged at 500×g for 5 minutes at 4° C. The supernatant was discarded, and the resulting cell pellet was resuspended in PBS and centrifuged at 500×g for 5 minutes at 4° C. The resulting pellet was resuspended in culture media containing Leibovitz's L-15 Medium (Gibco, cat. no. 11415064) supplemented with 20% fetal bovine serum and 10 mM HEPES (Gibco, cat. no. 15630106) and distributed into a 96 well plate. The cells were cultivated in an incubator at 28° C. with 5% CO₂ in a humidified atmosphere. For prevention of bacterial or fungal contamination, 100 U/ml Penicillin, 100 μg/ml Streptomycin (Gibco cat. no. 15140122), 1.25 μg/ml Amphotericin B (Gibco cat. no. 15290026) and 50 μg/ml Gentamicin (Gibco cat. no. 15710064) were added to the media during the first four weeks of culture growth. After four weeks of growth, the media contained Leibovitz's L-15 Medium (Gibco, cat. no. 11415064) supplemented with 20% fetal bovine serum, 10 mM HEPES (Gibco, cat. no. 15630106), 100 U/ml Penicillin and 100 μg/ml Streptomycin (Gibco cat. no. 15140122).

After initiation of the culture, the following culture regimen was performed. The cells were seeded at a cell concentration of ˜100,000 per well with 100 μl culture media. After three and five days, 50 μl was added to the culture. On day 7, 100-150 μl of the media was withdrawn and 50 μl fresh culture medium was added to the cells. This cultivation procedure was performed until the cells reached confluency and the culture was split.

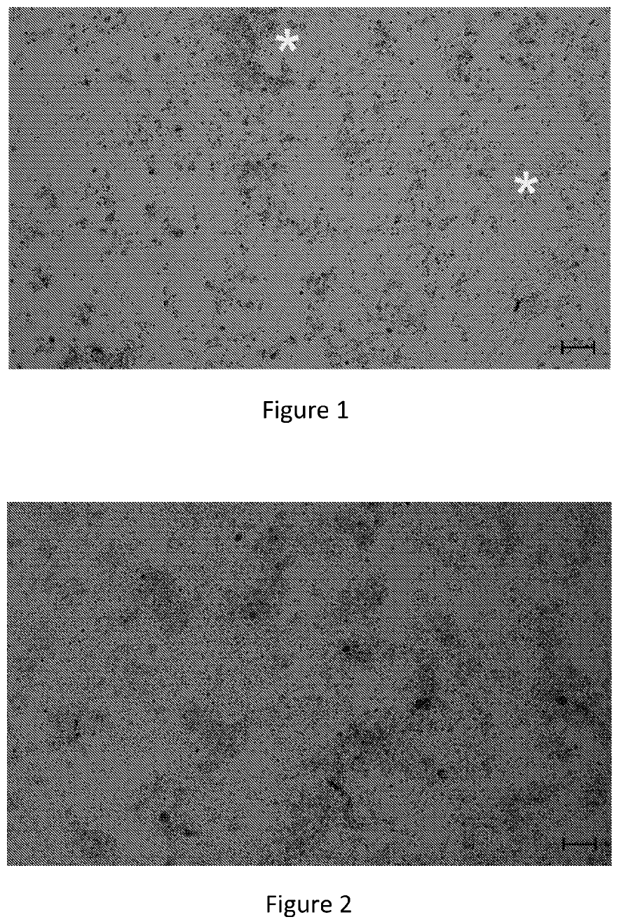

The cells were split because the culture contained a mixture of suspension cells and adherent cells (FIG. 1). First the culture media was resuspended and the resulting media/suspension cell mixture was distributed to two new wells. The remaining adherent cells were washed once with PBS and then detached with Trypsin/EDTA (Gibco cat.no. 12563011). The detached cells were resuspended in culture media and combined with the suspension cells. Unexpectedly, after one or two passages, no adherent cells were visible and the culture contained only suspension cells (FIG. 2). The suspension culture was split into two new wells.

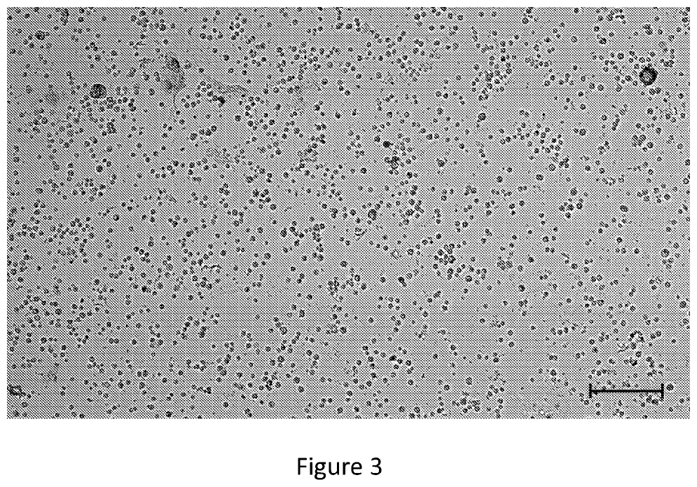

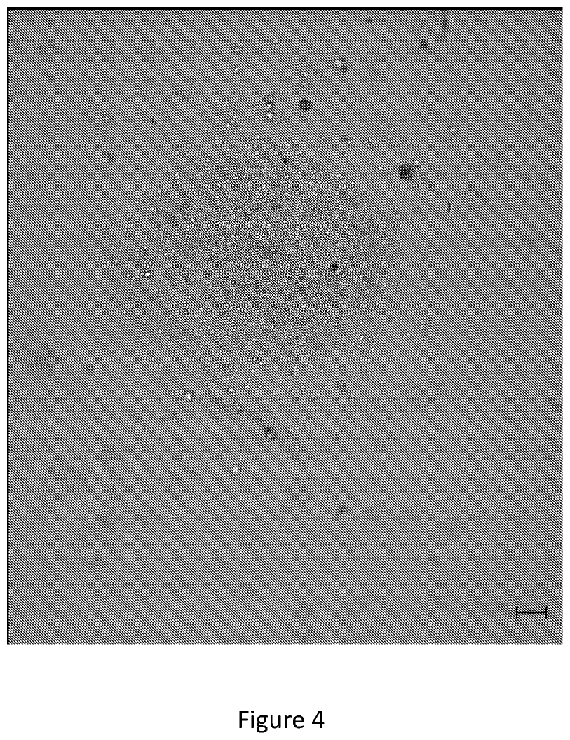

With prolonged culture using the abovementioned culture regimen, the cell culture became more and more homogenous with the majority of the cells exhibiting a round cell shape with a diameter between 5-8 μm (FIG. 3). The homogeneity of the culture became even more pronounced when suspension cells were cultivated in a round bottom plate which allows them to gather in the middle of the plate (FIG. 4). Advantageously, using this culture regimen, the cells were expanded for more than 6 months reaching expansion factors of between 15 and 20. Expansion factors were determined based on cell counts using a Neubauer chamber.

In follow-up experiments, suspension cells were treated with the epigenetic modifiers valproic acid and butyric acid. Valproic acid was used at concentrations of 30 μM, 100 μM, 300 μM, 1 mM and 3 mM; and butyric acid was used at concentrations of 1 μM, 10 μM, 50 μM, 100 μM and 500 μM. Suspension cells were exposed to either valproic acid or butyric acid once per week. An increase in proliferation was unexpectedly observed in both treatments compared to the standard culture described above. For valproic acid, an expansion factor of 8 was achieved within 6 weeks whereas for butyric acid, an expansion factor of 16 was reached within 4 weeks.

Expanded cells were cryopreserved with cryoconservation media containing Leibovitz's L-15 Medium (Gibco, cat. no. 11415064) supplemented with 10% fetal bovine serum and 7% ethylen glycol (ThermoFisher, cat. no. 29810). For cryoconservation, 1 million cells were pelleted at 500×g for 5 minutes at 4° C. and resuspended with cryopreservation media. The cells were placed in a Mr. Frosty cooling device which enables constant cooling of the samples. The Mr. Frosty cooling device was placed in a −80° C. freezer overnight before the cells were placed in a liquid-nitrogen tank for long-term storage.

Further investigations were performed. For example, the ability of the extracellular matrix molecules to support proliferation of suspension cells was investigated. To investigate whether collagen has a beneficial effect on proliferation, cell culture dishes were pre-coated with rat collagen type I solution (InSCREENeX Cat. no. INS-SU-1017). For this purpose, the collagen solution was added to the respective wells (50 μl per well in a 96 well plate; 200 μl per well in a 24 well plate; 400 μl per well in a 12 well plate; 600 μl per well in a 6 well plate), incubated for 2 h at 37° C. before the collagen solution was aspirated, the cell culture vessel washed with PBS and then the cell suspension transferred to the collagenized cell culture vessel. When the coated cell culture vessels were used for cultivation of the suspension cells, they supported the adherence of the cells, but counteracted the aim of creating a culture system for the expansion of sturgeon suspension cells.

The ability of Matrigel to support proliferation of suspension cells was also investigated. Matrigel is a solubilised basement membrane matrix secreted by Engelbreth-Holm-Swarm mouse sarcoma cells, and produced commercially by Corning Life Sciences. Matrigel is typically used for the expansion of adult stem cells, such as organoids. Suspension cells were centrifuged at 500×g for 5 minutes at 4° C. and the cells were resuspended in a mixture of 1:1 culture media with growth factor reduced Matrigel. This mixture was then used for the generation of a Matrigel dome in a 24 well plate. 60 μl of the cells/culture media/Matrigel mixture was pipetted in a 24 well plate and put in the incubator at 28° C., 5% CO₂ for 30 minutes to allow solidification of the Matrigel and dome formation. Afterwards the Matrigel dome was covered with an additional 100 μl of culture media. As the cells are contained within the Matrigel dome, the culture media can be easily aspirated and renewed without disturbing the cells. The media was renewed twice a week and proliferation of the cells was monitored microscopically. Unexpectedly, no proliferation was detected in any of the tested Matrigel domes (n=5).

### Cryopreservation of Fish Cells

Cells were cryopreserved in basal medium (0.13 M NaCl, 2.5 mM, KCl, 7.7 mM NaH₂PO₄, 0.7 mM KH₂PO₄, 0.9 mM, CaCl₂, 0.5 mM MgCl₂, 5.5 mM D-glucose, 0.09 mM sodium pyrvate, 0.5% bovine serum albumin) containing various amounts of four cryoprotectants: dimethylsulfoxide (DMSO), glycerol (Gly), 1,2-propanediol (PROH), and ethylene glycol (EG). After preservation in liquid nitrogen, cells were rapidly thawed and, if necessary, dissociated with 0.5% trypsin. The survival rates of frozen/thawed PGCs were assessed by ability to exclude trypanblue dye.

### Immortalisation of Cells Derived From Fish Tissues

Following 1-3 days of incubation at 37° C., confluent cultures are trypsinized and frozen, at 2-3×10⁶ cells per vial. To establish cell lines, cryopreserved fish cells (passage 0) are thawed and seeded into T75 flasks containing growth media. At 80% confluence, the cells are trypsinized and re-seeded onto 6-well culture dishes or small flasks (12.5 cm²) at a density of approximately 1×10⁵ cells per well/flask in growth media. Cultures are maintained under standard conditions (37° C., 5% CO₂ in a humidified atmosphere), and passaged serially when reaching confluence at a ratio of 1:3 or 1:4. Cells then reach a stage of “crisis” which is defined as when the majority of cells cease to divide further. Crisis is observed over passage 5-6 during a 3-week period. During crisis, culture medium is changed daily. Following crisis, over 95% of all cultures become spontaneously immortalized.

### Isolation, Characterisation and Ranking of Clonally-Derived Immortalised Cell Populations

Clonally-derived immortalised cell populations (CICPs) are obtained by limiting dilution to obtain a monoclonal cell population starting from a polyclonal mass of cells. A series of increasing dilutions of the parent (polyclonal) cell culture is made in order that aliquots of the suspension can then be distributed to wells at an average of 0.5 cells/well and incubated. CICPs are characterised by i) extent of staining by anti-DDX4/Vasa antibodies and ii) doubling time. CICPs are ranked by each metric separately and selection of CICPs with optimal growth and DDX4/Vasa signal are selected for expansion and large-scale cell banking.

### Differentiation of Clonally-Derived Fish Cell Populations to Oocytes/Mature Oocytes

Selected CICPs are cryo-revived and cultivated in growth media formulated to achieve a given level of viscosity and presence of supplements that favour in vitro differentiation of the CICPs into cells with morphology closely matching those described by Zelazowska et al (2007) as: Early previtellogenic (stage 1), Mid-previtellogenic (stage 2), Late-previtellogenic (stage 3), Vitellogenic (stage 4), and Postvitellogenic (stage 5), with these morphologic features being assessed by microscopy and staining procedures. Growth media supplements which favour in vitro differentiation into oocytes include epidermal growth factor (25 ng/mL), basic fibroblast growth factor (25 ng/ml), human chorionic gonadotropin (5 IU/mL), pregnant mare serum gonadotropin (2 IU/mL), glial cell line-derived neurotrophic factor (25 ng/ml) and leukemia inhibitory factor (25 ng/ml).

### Differentiation of Immature to Mature Oocytes

A given clonally-derived immortalised cell population is cultivated by passaging serially, when reaching confluence at a ratio of 1:3 or 1:4, for a given duration and cells are periodically monitored with respect to their size and morphology for progression through Mid-previtellogenic (stage 2), Late-previtellogenic (stage 3), Vitellogenic (stage 4), and Postvitellogenic (stage 5) staged of differentiation, with size and morphology enumerated against comparators. Upon transition from stage 1 to stage 2 the appearance of clear vesicles is observed in the cytoplasm, ingressing to the cell centre from the periphery of the cell. The nucleolus remains perinucleolar in stage 2. Upon transition to stage 2 a thin acidophilic zona radiata or primary envelope becomes visible. Follicular layers are also seen for the first time. Upon transition from stage 2 to stage 3 oocyte cell size is observed to increase and yolk granules become visible as a ring of deep eosinophilic inclusions in the cytoplasm. Upon transition from stage 3 to stage 4 the oocyte cell is larger and more hydrated, and the nucleus has migrated toward the periphery and tends to be in the process of dissolution. Upon transition from stage 4 to stage 5 the oocyte cell encompasses a large mass of yolk and a hydrated, swollen appearance. Samples of cells are withdrawn periodically and sacrificially analysed for the loss of biochemical markers of pluripotency, such as high levels of alkaline phosphatase or expression of genes such as dead end, grip2, plk3, gfra1a, and ednrba.

### REFERENCES

Ciba P, Schicktanz S, Anders E, Siegl E, Stielow A, Klink E, Kruse C. (2008). Long-term culture of a cell population from Siberian sturgeon (Acipenser baerii) head kidney. Fish Physiol Biochem. 34 (4): 367-72.
Geven EJW, Klaren PHM. (2017) The teleost head kidney: Integrating thyroid and immune signalling. Dev Comp Immunol. 66:73-83.
Streisinger G, Walker C, Dower N, Knauber D, Singer F (1981) Production of clones of homozygous diploid zebra fish (Brachydanio rerio). Nature 291:293-296.
Wong TT, Tesfamichael A, Collodi P. (2013). Production of zebrafish offspring from cultured female germline stem cells. PLOS One. 8 (5): e62660.
Xie X, Li P, Pšenička M, Ye H, Steinbach C, Li C, Wei Q. (2019) Optimization of In Vitro Culture Conditions of Sturgeon Germ Cells for Purpose of Surrogate Production. Animals (Basel) 9 (3): 106.
Zelazowska M, Kilarski W, Bilinski SM, Podder DD, Kloc M. Balbiani cytoplasm in oocytes of a primitive fish, the sturgeon Acipenser gueldenstaedtii, and its potential homology to the Balbiani body (mitochondrial cloud) of Xenopus laevis oocytes. Cell Tissue Res. 2007 July;329 (1): 137-45. doi: 10.1007/s00441-007-0403-9. Epub 2007 Mar. 16. PMID: 17364198.

1. An isolated stage 1 germ cell from a non-mammalian aquatic animal.

2. The isolated stage 1 germ cell of claim 1, wherein the germ cell is an oocyte.

3. An immortalised germ cell from a non-mammalian aquatic animal.

4. The immortalised germ cell of claim 3, wherein the germ cell is an oocyte.

5. The immortalised germ cell of claim 3, wherein the germ cell is a stage 1 oocyte.

6. The immortalised germ cell of claim 3, wherein the germ cell is a stage 2 oocyte.

7. The immortalised germ cell of claim 3, wherein the germ cell is a stage 3 oocyte, a stage 4 oocyte or a stage 5 oocyte.

8. The immortalised germ cell of claim 3, wherein the germ cell is a spermatozoa.

9. An immortalised primordial germ cell (PGC) from a non-mammalian aquatic animal.

10. The germ cell according to any one of claims 1-8 or the immortalised PGC according to claim 9, wherein the cell is a suspension cell.

11. An extract of a germ cell according to any one of claim 1-8 or 10 or an extract of an immortalised PGC according to claim 9 or claim 10.

12. A cosmetic product comprising a germ cell according to any one of claim 1-8 or 10, an immortalised PGC according to claim 9 or claim 10, or an extract according to claim 11.

13. A nutritional product comprising a germ cell according to any one of claim 1-8 or 10, an immortalised PGC according to claim 9 or claim 10, or an extract according to claim 11.

14. The nutritional product of claim 13, wherein one or more of the germ cells or immortalised PGCs are intact.

15. The nutritional product of claim 13 or claim 14, wherein one or more of the germ cells or immortalised PGCs are disrupted.

16. The nutritional product of any one of claims 13-15, wherein the germ cell(s), immortalised PGC(s) or extract are sugar-salted.

17. The nutritional product of any one of claims 13-16, wherein the germ cell(s), immortalised PGC(s) or extract are formulated in brine.

18. The nutritional product of any one of claims 13-17, wherein the germ cell(s), immortalised PGC(s) or extract are dried.

19. The nutritional product of any one of claims 13-18, wherein the germ cell(s), immortalised PGC(s) or extract are compressed.

20. The nutritional product of any one of claims 13-19, wherein the nutritional product is smoked.

21. The nutritional product of any one of claims 13-20, wherein the germ cell(s), immortalised PGC(s) or extract are within a bead or a pearl.

22. The nutritional product of claim 21, wherein the bead or pearl is a gel.

23. The nutritional product of claim 21 or claim 22, wherein the bead or pearl comprises sodium alginate.

24. The nutritional product of any one of claims 13-23, further comprising oil, optionally vegetable oil.

25. The nutritional product of any one of claims 13-24, wherein the nutritional product is canned.

26. The nutritional product of any one of claims 13-24, wherein the nutritional product is in a tube.

27. The nutritional product of any one of claims 13-26, wherein the nutritional product is spreadable.

28. The nutritional product of any one of claims 13-27, wherein the nutritional product is vacuum packed.

29. The nutritional product of any one of claims 13-28, wherein the nutritional product comprises one or more of bread, lemon juice, garlic and pepper.

30. A nutraceutical product comprising a germ cell according to any one of claim 1-8 or 10, an immortalised PGC according to claim 9 or claim 10, or an extract according to claim 11.

31. The extract according to claim 11, the cosmetic product according to claim 12, the nutritional product according to any one of claims claim 13-29, or the nutraceutical product according to claim 30, further comprising a salt.

32. An in vitro method of producing suspension germ cells from a non-mammalian aquatic animal, the method comprising:
(a) culturing a mixture of cells comprising germ cells from a non-mammalian aquatic animal in growth media until the mixture of cells reaches confluency;
(b) separating suspension cells from the cultured mixture of cells to provide a mixture of suspension cells; and
(c) optionally separating suspension germ cells from the mixture of suspension cells.

33. An in vitro method of producing suspension PGCs from a non-mammalian aquatic animal, the method comprising:
(a) culturing a mixture of cells comprising PGCs from a non-mammalian aquatic animal in growth media until the mixture of cells reaches confluency;
(b) separating suspension cells from the cultured mixture of cells to provide a mixture of suspension cells; and
(c) optionally separating suspension PGCs from the mixture of suspension cells.

34. The in vitro method of claim 32 or claim 33, wherein separating suspension germ cells from the mixture of suspension cells comprises screening for genetic markers.

35. The in vitro method according to claim 32 or claim 34, wherein the mixture of cells is a purified mixture of germ cells from a non-mammalian aquatic animal.

36. The in vitro method according to any one of claims 32-35, further comprising culturing the cells in the presence of an epigenetic modifier, optionally wherein the epigenetic modifier is selected from valproic acid and butyric acid.

37. The in vitro method according to any one of claim 32 or 34-36, wherein the germ cell is selected from a stage 1 germ cell, optionally a stage 1 oocyte, and a spermatozoa.

38. An in vitro method of producing isolated stage 1 oocytes from a non-mammalian aquatic animal, the method comprising:
(a) culturing a PGC from a non-mammalian aquatic animal to induce differentiation into germ cells; and optionally
(b) separating stage 1 oocytes from the PGCs.

39. An in vitro method of producing isolated spermatozoa from a non-mammalian aquatic animal, the method comprising:
(a) culturing a PGC from a non-mammalian aquatic animal to induce differentiation into germ cells; and optionally
(b) separating spermatozoa from the PGCs.

40. An in vitro method of producing an extract of an isolated stage 1 oocyte from a non-mammalian aquatic animal, the method comprising:
(a) culturing a PGC from a non-mammalian aquatic animal to induce differentiation into germ cells;
(b) optionally separating stage 1 oocytes from the PGCs; and
(c) processing the oocytes to provide an extract.

41. The in vitro method of claims 38-40, wherein the germ cells are separated from PGC(s) prior to separating oocytes from the PGCs.

42. The in vitro method of any one of claims 38-41, wherein the PGC is an immortalised PGC.

43. An in vitro method of producing isolated stage 1 oocytes from a non-mammalian aquatic animal, the method comprising culturing immortalised stage 1 oocytes to induce proliferation of stage 1 oocytes.

44. A method of producing a cosmetic product, the method comprising combining:
(a) a germ cell according to any one of claim 1-8 or 10, an immortalised PGC according to claim 9 or claim 10, or an extract according to claim 11 or 31; with
(b) ingredients for a cosmetic product.

45. A method of producing a nutritional product, the method comprising combining:
(a) a germ cell according to any one of claim 1-8 or 10, an immortalised PGC according to claim 9 or claim 10, or an extract according to claim 11 or 31; with
(b) ingredients for a nutritional product.

46. A method of producing a nutraceutical product, the method comprising combining:
(a) a germ cell according to any one of claim 1-8 or 10, an immortalised PGC according to claim 9 or claim 10, or an extract according to claim 11 or 31; with
(b) ingredients for a nutraceutical product.

47. The isolated stage 1 germ cell, the immortalised germ cell, the immortalised PGC, the extract, the cosmetic product, the nutritional product, the nutraceutical product, the in vitro method or the method of producing of any one of the preceding claims, wherein the non-mammalian aquatic animal is a fish.

48. The isolated stage 1 germ cell, the immortalised germ cell, the immortalised PGC, the extract, the cosmetic product, the nutritional product, the nutraceutical product, the in vitro method or the method of producing of claim 47, wherein the fish is selected from the group consisting of sturgeon, salmon, lumpfish, hake, trout, capelin, steelhead, whitefish, carp, pollock (e.g. Alaska pollock), herring (e.g. Atlantic herring), tobiko, imitation tobiko, cod (e.g. Atlantic cod), tuna, whitefish, catfish, shad, orange roughy, mullet, mako shark, perch (e.g. pike perch), jawless fish, armoured fish, spiny shark, cartilaginous fish, bony fish, ray-finned fish and lobe-finned fish.

49. The isolated stage 1 germ cell, the immortalised germ cell, the immortalised PGC, the extract, the cosmetic product, the nutritional product, the nutraceutical product, the in vitro method or the method of producing of claim 48, wherein the fish is selected from the group consisting of sturgeon, salmon, lumpfish, trout and cod (e.g. Atlantic cod).

50. The isolated stage 1 germ cell, the immortalised germ cell, the immortalised PGC, the extract, the cosmetic product, the nutritional product, the nutraceutical product, the in vitro method or the method of producing according to any one of claims 1-46, wherein the non-mammalian aquatic animal is an echinoderm, optionally wherein the echinoderm is a sea urchin or sea cucumber.

51. The isolated stage 1 germ cell, the immortalised germ cell, the immortalised PGC, the extract, the cosmetic product, the nutritional product, the nutraceutical product, the in vitro method or the method of producing according to any one of claims 1-46, wherein the non-mammalian aquatic animal is a crustacean, optionally wherein the crustacean in a crab or lobster.

52. Use of the germ cell according to any one of claim 1-8, 10 or 47-51 as an intermediate in the production of a higher stage oocyte, optionally wherein the higher stage oocyte is a stage 2 oocyte, a stage 3 oocyte, a stage 4 oocyte, or a stage 5 oocyte.

53. Use of the immortalised PGC according to any one of claim 9, 10 or 47-51 as an intermediate in the production of a germ cell, optionally an oocyte e.g. a stage 1 oocyte, a stage 2 oocyte, a stage 3 oocyte, a stage 4 oocyte, or a stage 5 oocyte.
